SEQUENCE LISTING (1 sequence)

SEQ ID NO: 1 - protein, 868 aa, Acidithiobacillus ferrooxidans
MAHVVILGGGTGGMPAAYEMKEALGSGHEVTLISANDYFQFVPSNPWVGVGWKERDDITFMAHVVILGAGTGGMPAAYEMKEALGSGHEVTLISANDYFQFVPSNPWVGVGWKERDDIAFPIRHYVERKGIHFVAQSAERIDAEAQNITLADGSTVHYDYLMITAGPKLAFENVPGSPHPPIRHYVERKGIHFIAQSAEQIDAEAQNITLADGNTVHYDYLMIATGPKLAFENVPGSDPHEGPVQSICTVDHAERAFAEYQALLREPGPIAIGAMAGASCFGPAYEYAMIVASDLKKRGMEGPVQSICTVDHAERAFAEYQALLREPGPIVIGAMAGASCFGPAYEYAMIVASDLKKRGMRDKIPSFTFITSEPYLGHLGIQGVGDSKGILTKGLKEEGIEAYTNCKVTKVEDNKMYVTQRDKIPSFTFITSEPYIGHLGIQGVGDSKGILTKGLKEEGIEAYTNCKVTKVEDNKMYVTQVDEKGETIKEMVLPVKLGMMIPAFKGVPAVAGVEGLCNPGGFVLVDEHQRSKKYANIFAAVDEKGETIKEMVLPVKFGMMIPAFKGVPAVAGVEGLCNPGGFVLVDEHQRSKKYANIFAAGIAIAIPPVETTPVPTGAPKTGYMIESMVSAAVHNIKADLEGRKGEQTMGTWNAVCFADMGIAIAIPPVETTPVPTGAPKTGYMIESMVSAAVHNIKADLEGRKGEQTMGTWNAVCFADMGDRGAAFIALPQLKPRKVDVFAYGRWVHLAKVAFEKYFIRKMKIGVSEPFYEKVLFKMMGGDRGAAFIALPQLKPRKVDVFAYGRWVHLAKVAFEKYFIRKMKMGVSEPFYEKVLFKMMGITRLKEEDAHRKASITRAKEEDTHRKAS

(19) United States
(12) Patent Application Publication
(10) Pub. No.: US 2016/0039697 A1
(43) Pub. Date: Feb. 11, 2016

(54) METHOD OF MITIGATING HYDROGEN SULFIDE OR MERCAPTAN CONTAMINATION WITH AN ENZYME BASED SCAVENGER

(71) Applicant: Baker Hughes Incorporated, Houston, TX (US)

(72) Inventors: Prasad D. Dhulipala, Austin, TX (US); Charles David Armstrong, Tomball, TX (US); Qi Qu, Spring, TX (US)

(21) Appl. No.: 14/454,417

(22) Filed: Aug. 7, 2014

(51) Int. Cl. C02F 3/34; C12N 9/02; B01D 53/84; B01D 53/52; B01D 53/48

(52) U.S. Cl. CPC C02F 3/342; B01D 53/52; B01D 53/48; B01D 53/84; C12N 9/0051; C02F 2303/02; C02F 2303/08; C02F 2101/101

(57) ABSTRACT

Hydrogen sulfide and mercaptans may be catalytically removed from a fluid stream by introducing to the fluid stream a sulfide quinone reductase (SQR) enzyme.

### FIELD OF THE DISCLOSURE

The disclosure relates to a method of mitigating hydrogen sulfide and/or mercaptans within a liquid or gaseous stream by contacting the liquid or gaseous stream with a catalytically effective amount of a Sulfide Quinone Reductase enzyme.

### BACKGROUND OF THE DISCLOSURE

Hydrogen sulfide and mercaptans are present in underground water removed with crude oil, in crude oil itself, in natural gases and in gases associated with underground water and crude oil. Hydrogen sulfide and mercaptans are characterized by highly noxious odors and typically are highly corrosive. Uncontrolled emissions of hydrogen sulfide give rise to severe health hazards. The presence of hydrogen sulfide and mercaptans is further objectionable because they often react with desirable hydrocarbons as well as fuel system components.

Treatments for removal of hydrogen sulfide and mercaptans from hydrocarbons and other substrates include the use of various reactive organic compounds as scavengers. For example, U.S. Pat. No. 6,063,346 discloses the use of maleimides, formaldehydes, amines, carboxamides, alkylcarboxyl-azo compounds and cumine-peroxide compounds for the removal of hydrogen sulfide and mercaptans. Further, U.S. Pat. No. 5,128,049 discloses the use of certain morpholino and amino derivatives for the removal of hydrogen sulfide. In addition, U.S. Pat. Nos. 8,022,017; 7,264,786; 6,063,346 and 5,128,049 disclose the use of triazines to remove hydrogen sulfide. Such scavengers however are often considered to be undesirable since they are not environmentally preferable.

Since the generation of hydrogen sulfide and mercaptans is often encountered throughout drilling, production, transport, storage and processing of crude oil as well as underground water, there is a need for a method of mitigating the formation of hydrogen sulfide and mercaptans and the deleterious effects created by hydrogen sulfide and mercaptans in an environmentally preferable manner.

It should be understood that the above-described discussion is provided for illustrative purposes only and is not intended to limit the scope or subject matter of the appended claims or those of any related patent application or patent. Thus, none of the appended claims or claims of any related application or patent should be limited by the above discussion or construed to address, include or exclude each or any of the above-cited features or disadvantages merely because of the mention thereof herein.

Accordingly, there exists a need for an environmentally friendly or “green” method for mitigating hydrogen sulfide and/or mercaptans within a liquid or gaseous stream having one or more of the attributes or capabilities described or shown in, or as may be apparent from, the other portions of this patent.

### SUMMARY OF THE DISCLOSURE

In an embodiment of the disclosure, a method of scavenging hydrogen sulfide and/or mercaptans from a liquid or gaseous stream is provided. In the method, a catalytically effective amount of a Sulfide Quinone Reductase (SQR) enzyme is brought into contact with the liquid or gaseous stream.

In another embodiment of the disclosure, a method of scavenging hydrogen sulfide and/or mercaptans from a liquid or gaseous stream within a hydrocarbon producing reservoir is provided wherein a catalytically effective amount of a SQR enzyme is brought into contact with the liquid or gaseous stream.

In another embodiment of the disclosure, a method of scavenging hydrogen sulfide and/or mercaptans from a liquid or gaseous stream within a storage vessel is provided wherein a catalytically effective amount of a SQR enzyme is brought into contact with the liquid or gaseous stream.

In another embodiment of the disclosure, a method of scavenging hydrogen sulfide and/or mercaptans from an unrefined or refined hydrocarbon product derived from petroleum or from the liquefaction of coal is provided. In this method, a catalytically effective amount of a SQR enzyme is brought into contact with the unrefined or refined hydrocarbon product.

In another embodiment, a method of scavenging hydrogen sulfide and/or mercaptans from a liquid or gaseous stream is provided. In this method, a liquid or gaseous stream is brought into contact with a SQR enzyme. Hydrogen sulfide and/or mercaptans are then oxidized to sulfur containing oxidation product.

In another embodiment, a method for catalytically mitigating hydrogen sulfide or mercaptans or both hydrogen sulfide and mercaptans from a liquid or gaseous stream in a hydrocarbon producing reservoir is provided. In this method, a catalytically effective amount of a SQR enzyme is pumped into the reservoir.

Accordingly, the present disclosure includes features and advantages which mitigate hydrogen sulfide and/or mercaptans in a liquid or gaseous stream. Characteristics and advantages of the present disclosure described above and additional features and benefits will be readily apparent to those skilled in the art upon consideration of the following detailed description of various embodiments and referring to the accompanying figures.

### BRIEF DESCRIPTION OF THE DRAWINGS

The following figures are part of the present specification, included to demonstrate certain aspects of various embodiments of this disclosure and are referenced in the detailed description herein:

FIG. 1 depicts an amino acid sequence of sulfide quinone reductase (SQR) enzyme variants used in the disclosure as a hydrogen sulfide scavenger.

FIG. 2 illustrates the effectiveness of a SQR enzyme as a hydrogen sulfide scavenger in soured well water.

FIG. 3 illustrates reduction of H₂S/sulfide by a SQR enzyme from a sodium sulfide solution.

FIG. 4 illustrates the functionality of a SQR enzyme as a hydrogen sulfide barrier.

FIG. 5 demonstrates the effect of pH on the activity of a SQR enzyme on the reduction of hydrogen sulfide.

FIG. 6 demonstrates the effect of temperature on the activity of a SQR enzyme.

FIG. 7 illustrates the tolerance of a SQR enzyme to salt.

FIG. 8 illustrates the reduction of hydrogen sulfide from head space using a SRQ enzyme.

### DETAILED DESCRIPTION OF THE PREFERRED EMBODIMENTS

Characteristics and advantages of the present disclosure and additional features and benefits will be readily apparent to those skilled in the art upon consideration of the following detailed description of exemplary embodiments of the present disclosure. It should be understood that the description herein, being of example embodiments, are not intended to limit the claims of this patent or any patent or patent application claiming priority hereto. Many changes may be made to the particular embodiments and details disclosed herein without departing from such spirit and scope.

As used herein and throughout various portions (and headings) of this patent application, the terms “disclosure”, “present disclosure” and variations thereof are not intended to mean every possible embodiment encompassed by this disclosure or any particular claim(s). Thus, the subject matter of each such reference should not be considered as necessary for, or part of, every embodiment hereof or of any particular claim(s) merely because of such reference. Also, the terms “including” and “comprising” are used herein and in the appended claims in an open-ended fashion, and thus should be interpreted to mean “including, but not limited to . . . .”

In the present disclosure, a liquid or gaseous stream rendered “sour” by the presence of sulfhydryl compounds is brought into contact with a catalytically effective amount of a sulfide quinone reductase (SQR) enzyme. The liquid or gaseous stream may be an aqueous substrate or a hydrocarbon substrate.

As used herein, the term “mercaptan” shall include alkyl mercaptans and thiols of the formula R—SH where R is an unsubstituted or substituted alkyl, thiol carboxylic acids and dithio acids.

As used herein, the term “aqueous substrate” shall refer to any “sour” aqueous substrate, including waste water streams in transit to or from municipal waste water treatment facilities, tanning facilities, and the like. The term “hydrocarbon substrate” is meant to include unrefined and refined hydrocarbon products, including natural gas, derived from petroleum or from the liquefaction of coal, both of which contain hydrogen sulfide or other sulfur-containing compounds. Thus, particularly for petroleum-based fuels, the term “hydrocarbon substrate” includes, but is not limited to, wellhead condensate as well as crude oil which may be contained in storage facilities at the producing field. “Hydrocarbon substrate” also includes the same materials transported from those facilities by barges, pipelines, tankers, or trucks to refinery storage tanks, or, alternately, transported directly from the producing facilities through pipelines to the refinery storage tanks The term “hydrocarbon substrate” also includes refined products, interim and final, produced in a refinery, including distillates such as gasoline, distillate fuels, oils, and residual fuels and to vapors produced by the foregoing materials. The method defined herein is therefore applicable to a wide variety of fluid streams, including liquefied petroleum gas as well as crude oil and petroleum residual fuel, heating oil, etc. In addition, the method is applicable to gaseous hydrocarbon streams. For instance, the composite may be contacted with wet or dry gaseous mixtures of hydrogen sulfide and/or mercaptan and hydrocarbon vapors, such as is found, for instance, in natural gas or obtained in the drilling, removal from the ground, storage, transport, and processing of crude oil.

The method disclosed herein has particular applicability in the removal of hydrogen sulfide and mercaptans of the formula R—SH wherein R is an alkyl group having from 1 to 40 carbon atoms and preferably from 1 to 20 carbon atoms, most preferably from 1 to 6 carbon. Such mercaptans are especially desirable for removal in light of their noxious odors and corrosive nature.

The sulfide quinone reductase (SQR) enzyme used as a hydrogen sulfide scavenger in the method disclosed herein may originate from various organisms. The SQR enzyme prevents the formation of hydrogen sulfide and mercaptans. The harm from such materials may be seen within the reservoir, in transport pipes as well as in storage areas and containers for fluids produced from the reservoir. In addition to contributing to corrosion of metals used during production, transport and/or storage of produced fluids, the presence of hydrogen sulfide and mercaptans further causes reservoir souring and presents a danger to oilfield personnel.

The dangers of hydrogen sulfide and mercaptans are mitigated by the addition of the SQR enzyme to the liquid or gaseous stream. The addition of SQR enzyme to the liquid or gaseous stream is believed to catalytically attack hydrogen sulfide and precursors of hydrogen sulfide. It further prevents the formation of hydrogen sulfide and mercaptans. It is believed that the SQR enzyme catalyzes the oxidative breakdown of hydrogen sulfide and mercaptans to sulfur containing oxidation product. The oxidation product could be elemental sulfur, a sulfite, a polysulfide, etc.

The SQR enzyme may remain stable and be effective at temperatures in excess of 205° F. Typically, the SQR enzyme is introduced into a liquid or gaseous substrate maintained at a temperature between from about 25 to about 180° F., more typically between from about 100 to about 150° C.

The amount of SQR enzyme added to the liquid or gaseous stream is that amount sufficient to effectuate the desired result over a sustained period of time and thus is dependent on the amount of the hydrogen sulfide and/or mercaptan in the medium being treated. In general, the amount of the SQR enzyme added to the medium is at least an effective scavenging amount, for example, from about 0.05 ppm to about 2,000 ppm or more, preferably from about 20 to about 1,200 ppm, and more preferably from about 100 to about 400 ppm of hydrogen sulfide and/or mercaptan.

The SQR enzyme is particularly effective in the treatment of liquid or gaseous streams having a pH between from about 5.5 to about 7.5.

Since SQR catalytically breaks down hydrogen sulfide and mercaptans, it may be used over an extended period of time (until it is no longer stable) without being replenished. In addition, the use of SQR inflicts reduced, minimal, or no harm to an aqueous or hydrocarbon substrate.

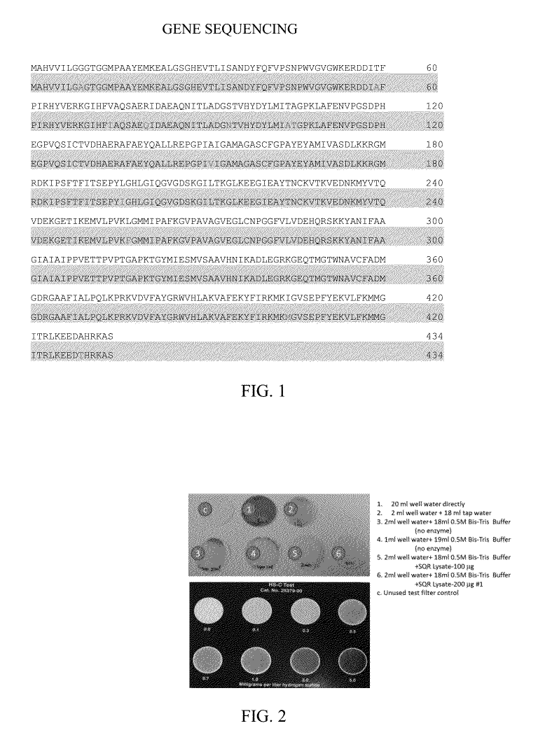

In a preferred embodiment, the nucleotide sequence encoding the SQR enzyme may be derived from a gram negative, acidophilic and thermophilic bacterium, such as Acidithobacillus ferroxidans, Metallospora cuprina and Metallospora sedula, using polymerase chain reaction (PCR) amplification. An amino acid sequence of SQR reductase enzyme variants Acidithiobacillus ferroxidans is set forth in FIG. 1. The SQR gene sequence was amplified using A. ferroxidans genomic DNA and was cloned in a protein expression vector. A homology may be similar for other SQR enzymes depending on originating organisms.

The SQR enzyme may be added to any aqueous or nonaqueous medium containing hydrogen sulfide and/or mercaptans where sulfides are sought to be mitigated. Such media include wet gaseous mediums containing water vapors and/or hydrocarbon vapors. Thus, the method disclosed herein is useful in controlling hydrogen sulfide and/or mercaptans in water systems, oil and gas production and storage systems, and other similar systems.

Generally, for industrial or commercial use, the SQR enzyme may be contacted with a stream containing the hydrogen sulfide or mercaptans for removal. Contact can occur in a variety of containers, such as a process or transport line, a separate stirred or non-stirred container or other vessels such as scrubbers or strippers. Further, the SQR enzyme may be added via surface or downhole equipment or at any time in the process stream in recovering crude oil so as to remove the noxious quality and corrosive nature of the hydrogen sulfide and mercaptans in the processing system.

In general, the SQR enzyme is injected into or otherwise brought into intimate contact with the liquid hydrocarbon, hydrogen sulfide and/or mercaptan and, when present, water and/or solvent in any convenient manner. The SQR enzyme has particular usefulness where the liquid or gaseous stream being treated is sour well water.

With emissions from a residual fuel oil, the SQR enzyme may be stirred into the fuel oil. When used with a natural gas, the natural gas may be scrubbed with an aqueous or nonaqueous solution containing the SQR enzyme. Additionally, when the natural gas, as it often does, contains water vapors, the SQR enzyme may be injected into a stream of the gas moving within a conduit. In such case, when the water vapors are removed from the natural gas as a liquid, the product resulting from the catalytic reaction of the hydrogen sulfide may then be removed.

In another embodiment, the liquid or gaseous stream being treated with the SQR enzyme may be sea water or brine. The liquid or gaseous stream being treated may be within a hydrocarbon producing reservoir or within a storage vessel. Further, the liquid or gaseous stream may be an unrefined or refined hydrocarbon product derived from petroleum or from the liquefaction of coal. Further, the liquid or gaseous stream may be a wet or dry gaseous mixture or hydrocarbon vapors.

The SQR enzyme may be added neat or diluted with water or solvent and may be formulated or blended with other suitable materials or additives. In a preferred embodiment, the SQR enzyme is included in a brine (such as a saturated potassium chloride or sodium chloride solution), salt water or fresh water. The diluent is selected in order for the SQR enzyme to be soluble both in the diluents and in the feed stream. For liquid systems, suitable solvents for dissolving the scavenger include polar and non-polar solvents such as water, alcohols, esters, benzene and benzene derivatives. The SQR enzyme may further be advantageously employed in liquefied gas and foamed gas carrier fluids, such as liquid CO₂, CO₂/N₂, and foamed N₂ in CO₂ based systems.

Well treatment compositions containing the SQR enzyme may further be gelled or non-gelled. Suitable carrier fluids include or may be used in combination with fluids have gelling agents, cross-linking agents, gel breakers, surfactants, foaming agents, demulsifiers, buffers, clay stabilizers, acids, or mixtures thereof. The SQR enzyme may be used in any well treatment operation where the presence of hydrogen sulfide and/or mercaptans may be encountered. As such, the SQR enzyme may be a component of a fracturing fluid (with or without the presence of a proppant), an acidizing fluid, drilling fluid, completion fluid, etc. In addition, the SQR enzyme may be used during the transport, storage and/or processing of oil or gas to address issues raised by the presence of hydrogen sulfide and/or mercaptans.

Preferred embodiments of the present disclosure thus offer advantages over the prior art and are well adapted to carry out one or more of the objects of this disclosure. However, the present disclosure does not require each of the components and acts described above and are in no way limited to the above-described embodiments or methods of operation. Any one or more of the above components, features and processes may be employed in any suitable configuration without inclusion of other such components, features and processes. Moreover, the present disclosure includes additional features, capabilities, functions, methods, uses and applications that have not been specifically addressed herein but are, or will become, apparent from the description herein, the appended drawings and claims.

All percentages set forth in the Examples are given in terms of weight units except as may otherwise be indicated.

The SQR enzyme used in the Examples had the amino acid sequence set forth in FIG. 1 and was prepared as discussed in the paragraphs above.

### EXAMPLES

### Example 1

This example illustrates the reduction of hydrogen sulfide in enzyme treated samples. Referring to FIG. 2, 20 ml of sour well water directly or the well water diluted 10 times using tap water (2) or 0.05M Bis-tris buffer (3) or well water diluted 20 times with 0.05 M Bis-Tris buffer (4) was used to measure hydrogen sulfide concentration. 100 μg or 200 μg of SQR crude lysate was added to the samples diluted 10 times with 0.05 M Bis-tris buffer and hydrogen sulfide measurements were performed after 30 min incubation to evaluate enzyme performance. A comparison of the results with a reference card supplied with the HACH filter assay kit revealed reduction of hydrogen sulfide in enzyme treated samples (5 and 6).

### Example 2

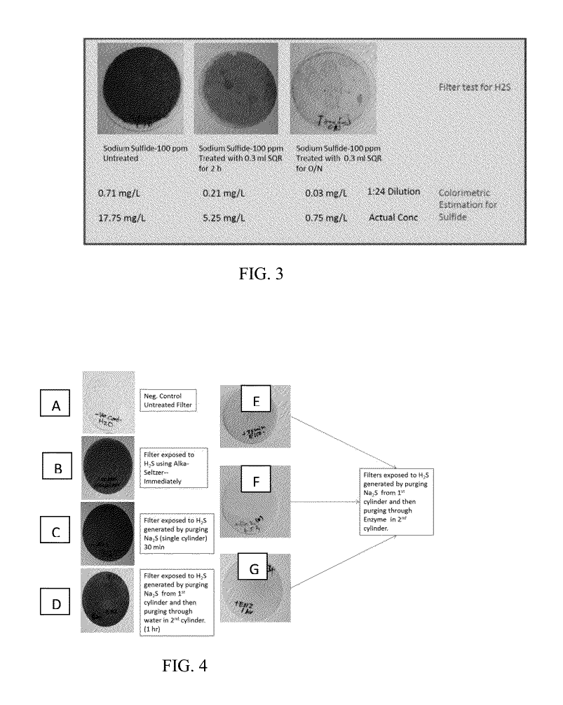

This example illustrates the reduction of H₂S sulfide by a SQR enzyme from a sodium sulfide solution. Referring to FIG. 3, sodium sulfide solution was used as the standard to generate hydrogen sulfide to test the SQR enzyme. 100 ppm of sulfide solution was prepared from sodium sulfide.9H₂O in water and equal amount of 0.1 M Bis-Tris buffer, pH 6 was added. 100 ml of this solution was incubated with 1 ml of SQR enzyme lysate for 2 h or 18 h. Sulfide concentrations were measured by HACH filter assay or by HACH colorimetric method. The results in FIG. 3 show that treatment of sulfide solution with SQR reduced sulfide to 30% in 2 hours and to 6% in 18 hours.

### Example 3

This Example illustrates the use of the SQR enzyme as a barrier to hydrogen sulfide. Referring to FIG. 4, an unreacted filter was used as negative control (A) and 100 ppm Na₂S solution was taken in to a bottle and exposed to filter (B), 150 ml of 100 ppm Na₂S solution was taken in a cylinder and purged with air. The air from the other end of the cylinder was collected on to filter paper (C). 150 ml of 100 ppm Na₂S solution was taken in a first cylinder and purged with air. The air from the other end of the cylinder was purged through water in the second cylinder and the air from the other end of the 2^{nd} cylinder was collected onto filter paper (D). Both C and D were taken as minus enzyme controls. To evaluate enzyme efficiency, the second cylinder was filled with 100 ml of 0.05M Bis-Tris buffer containing 2 ml of enzyme lysate and air was purged similar to C or D. The air from second cylinder was collected on the filter in 3 independent experiments (E, F, and G). Results show that the filters remained light blue indicating that there is no or very slight hydrogen sulfide in the air coming out from the enzyme cylinder.

### Example 4

This Example illustrates the effect of pH on the activity of the SQR enzyme. 0.1 M Bis-tris buffers with pH 4, 5, 6, 7, 8, 9 and 10 were prepared. 100 ppm sodium sulfide solution was prepared in water. To 25 ml of 100 ppm sodium sulfide, 25 ml of 0.1 M Bis-Tris buffer was added and mixed well. To this was added 0.5 ml of SQR enzyme lysate and the reactions were incubated for 18 hours. The sulfide concentrations were measured by HACH colorimetric method. FIG. 5 shows the optimal pH for the SQR enzyme to be at 6 and 7 where it reduced the maximum amount of sulfide.

### Example 5

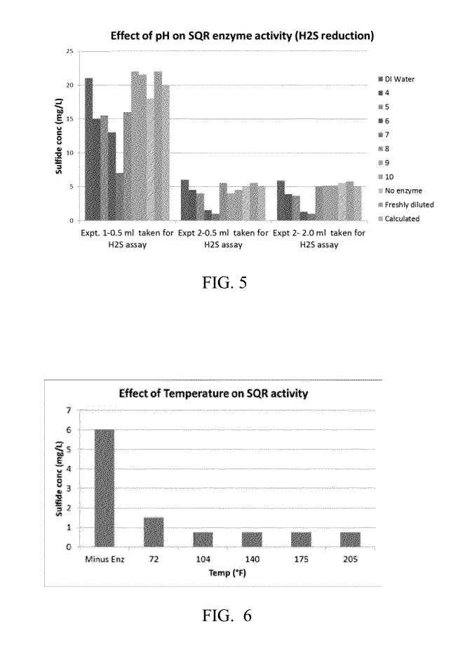

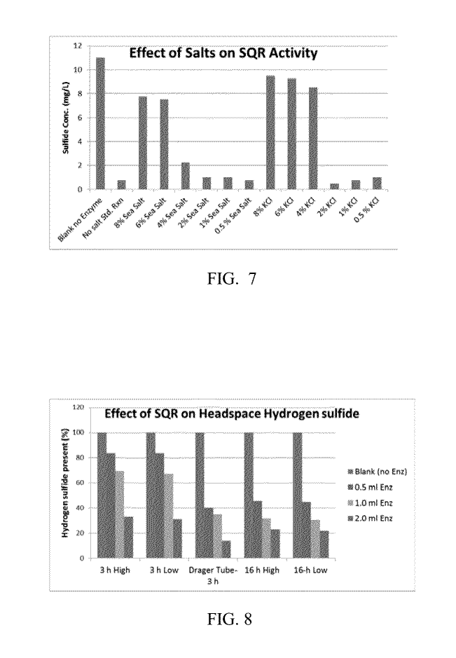

This Example illustrates the resistance to high temperature of the SQR enzyme. 100 ppm sodium sulfide solution was prepared in water. To 25 ml of 100 ppm sodium sulfide, 25 ml of 0.1 M Bis-Tris buffer was added and mixed well. To this 0.5 ml of SQR enzyme lysate was added and the reactions were incubated for 18 hours at different temperatures. The sulfide concentrations were measured by HACH colorimetric method. The results set forth in FIG. 6 show the SQR enzyme to exhibit good activity at all temperatures tested and that sulfide was reduced 12.5% from 104° F. to 205° F.

### Example 6

This Example illustrates the tolerance of the SQR enzyme to salt concentrations present in brine. 100 ppm sodium sulfide solution was prepared in water containing 1% to 16% sea salt or potassium chloride. To 25 ml of 100 ppm sodium sulfide containing salts, 25 ml of 0.1 M Bis-Tris buffer was added and mixed well. To this 0.5 ml of SQR enzyme lysate was added and the reactions were incubated for 18 hours at different temperatures. The sulfide concentrations were measured by HACH colorimetric method. The results illustrated in FIG. 6 show that the SQR enzyme had tolerance to up to 4% sea salt and 2% potassium chloride.

### Example 7

This Example illustrates reduction of hydrogen sulfide gas with the SQR enzyme from head space. 100 ppm sodium sulfide solution was prepared in water containing in 0.05 M Bis Tris buffer, pH 7.0. In eight 200 ml injection bottles, 90 ml of 100 ppm sulfide solution was taken and sealed airtight with septum. The bottles were divided into 2 sets of 4. First set bottles 1-4 were allowed to stand for 1.5 hours to reach equilibrium of hydrogen sulfide in head space. Initial hydrogen sulfide concentrations were measured by a Micro Gas Chromatography method. To bottle 1, no enzyme was added. To bottle 2 0.5 ml, to bottle 3—1 ml and to bottle 4 two ml enzyme lysates were added. The reactions were incubated for 16 hours. The sulfide concentrations were measured by Micro GC. Similarly second set of bottles 5-8 were allowed to stand for 16 hours to reach equilibrium of hydrogen sulfide in head space. Initial hydrogen sulfide concentrations were measured by a Micro Gas Chromatography method. To bottle 5, no enzyme was added. To bottle 6—0.5 ml, to bottle 7—1 ml and to bottle 8—w ml enzyme lysates were added. The reactions were incubated for 3 hours. The sulfide concentrations were measured by Micro GC. Low and High in FIG. 8 represent the two columns with low and high sensitivities that give accurate hydrogen sulfide concentrations. The concentration of hydrogen sulfide was also measured by Dragger tubes which confirmed the Micro GC data.

The methods that may be described above or claimed herein and any other methods which may fall within the scope of the appended claims can be performed in any desired suitable order and are not necessarily limited to any sequence described herein or as may be listed in the appended claims. Further, the methods of the present disclosure do not necessarily require use of the particular embodiments shown and described herein, but are equally applicable with any other suitable structure, form and configuration of components.

While exemplary embodiments of the disclosure have been shown and described, many variations, modifications and/or changes of the system, apparatus and methods of the present disclosure, such as in the components, details of construction and operation, arrangement of parts and/or methods of use, are possible, contemplated by the patent applicant(s), within the scope of the appended claims, and may be made and used by one of ordinary skill in the art without departing from the spirit or teachings of the disclosure and scope of appended claims. Thus, all matter herein set forth or shown in the accompanying drawings should be interpreted as illustrative, and the scope of the disclosure and the appended claims should not be limited to the embodiments described and shown herein.

What is claimed is:

1. A method of scavenging hydrogen sulfide and/or mercaptans from a liquid or gaseous stream which comprises bringing the stream into contact with a catalytically effective amount of a sulfide quinone reductase enzyme.

2. The method of claim 1, wherein the nucleotide sequence encoding the sulfide quinone reductase enzyme is derived from Acidithobacillus ferroxidans.

3. The method of claim 1, wherein the sulfide quinone reductase enzyme has the amino acid sequence set forth in FIG. 1.

4. The method of claim 1, wherein the liquid or gaseous stream is sour well water.

5. The method of claim 1, wherein the liquid or gaseous stream is sea water or brine.

6. The method of claim 1, wherein the liquid or gaseous stream has a pH between from about 5.5 to about 7.5.

7. The method of claim 1, wherein the sulfide quinone reductase enzyme is effective at a temperature up to about 205° F.

8. The method of claim 1, wherein the liquid or gaseous stream is within a hydrocarbon producing reservoir.

9. The method of claim 1, wherein the liquid or gaseous stream is within an unrefined or refined hydrocarbon product derived from petroleum or from the liquefaction of coal.

10. The method of claim 1, wherein the liquid or gaseous stream is a wet or dry gaseous mixture or hydrocarbon vapors.

11. A method of scavenging hydrogen sulfide and/or mercaptans from a liquid or gaseous stream which comprises:
(a) contacting the liquid or gaseous stream with a sulfide quinone reductase enzyme; and
(b) Oxidizing hydrogen sulfide and/or mercaptans to a sulfur containing oxidation product.

12. The method of claim 11, wherein the liquid or gaseous stream is within a hydrocarbon producing reservoir.

13. The method of claim 11, wherein the liquid or gaseous stream is within a storage vessel.

14. The method of claim 11, wherein the liquid or gaseous stream is an unrefined or refined hydrocarbon product derived from petroleum or from the liquefaction of coal.

15. The method of claim 11, wherein the nucleotide sequence encoding the sulfide quinone reductase enzyme is derived from Acidithobacillus ferroxidans.

16. The method of claim 11, wherein the sulfide quinone reductase enzyme has the amino acid sequence set forth in FIG. 1.

17. A method for catalytically mitigating hydrogen sulfide or mercaptans or both hydrogen sulfide and mercaptans from an aqueous or gaseous stream in a hydrocarbon producing reservoir comprising pumping into the reservoir a catalytically effective amount of a sulfide quinone reductase enzyme.

18. The method of claim 17, wherein the sulfide quinone reductase enzyme inhibits the formation of hydrogen sulfide, mercaptans or a combination of hydrogen sulfide and mercaptans.

19. The method of claim 17, wherein the sulfide quinone reductase enzyme removes hydrogen sulfide, mercaptans or a combination of hydrogen sulfide and mercaptans from the liquid or gaseous stream within the reservoir.

20. The method of claim 17, wherein the sulfide quinone reductase enzyme has the amino acid sequence set forth in FIG. 1.
